(19) United States
(12) Patent Application Publication
(10) Pub. No.: US 2016/0010851 A1
(43) Pub. Date: Jan. 14, 2016

(54) THERMOSIPHON ESTERIFIER STEAM REUSE

(71) Applicant: INVISTA NOTH AMERICA S.a.r.l., Wilmington, DE (US)

(72) Inventors: Robert Edward NEATE, Gusiborough (GB); Clive Alexander Hamilton, Swainby (GB); Robert E. Stilson, Wilmington, NC (US); George Malcolm Williamson, Wilmington, NC (US); Jonathan D. Runnacles, Stockton-on-Tees (GB)

(21) Appl. No.: 14/854,815

(22) Filed: Sep. 15, 2015

Related U.S. Application Data
(63) Continuation-in-part of application No. PCT/US2014/028774, filed on Mar. 14, 2014.
(60) Provisional application No. 61/792,164, filed on Mar. 15, 2013.

(51) Int. Cl. F22B 1/16; C07C 67/08

(52) U.S. Cl. CPC F22B 1/16; C07C 67/08

(57) ABSTRACT

The invention relates to recycling steam at elevated temperature and/or pressure generated within a thermosiphon esterifier design comprising a riser baffle in the vapor separator. Advantageously, the thermosiphon esterifier design can provide steam that can be employed in various additional processes and can thus provide an overall energy savings in operating the thermosiphon esterifier. Methods of using the steam are also described.

### FIELD OF THE INVENTION

The invention is related to a process for recycling waste heat from a novel thermosiphon esterifier design and to systems in which such processes can be implemented.

### BACKGROUND OF THE INVENTION

Polyethylene terephthalate) (PET) resins are widely produced and used, for example, in the form of fibers and in the form of bottle resin. PET is commonly used in the production of beverage and food containers, thermoforming applications, textiles, and as engineering resins. PET is a polymer based on the monomer unit bis-β-hydroxyterephthalate, which is commonly formed from ethylene glycol and terephthalic acid (or dimethyl terephthalate).

The manufacturing process for PET is generally conducted within a series of melt phase reactors, which may include an esterifier, up flow pre-polymerizer (UFPP) and finisher. These reactors typically operate at temperatures above 270° C., while the operating pressure reduces from super-atmospheric pressure in the first reactor (esterifier) to nearly full vacuum in the final reactor (finisher).

The raw materials for PET production are ethylene glycol and phthalic acids. The phthalic acids are typically 100% terephthalic acid for the production of polyester fibers, but may contain up to 5% isophthalic acid for bottle resins. Catalysts and other additives may be added to the process at any point, but are normally injected at some point before the first tray of the UFPP. In the esterifier, ethylene glycol is reacted with terephthalic acid via an esterification reaction to form an oligomer and water vapor as a byproduct. The oligomer is then polymerized in the UFPP and finisher to form the PET polymer product with ethylene glycol and water as byproducts.

Although both esterification and polymerization can occur to some extent in each of the reactors, typically, 85-95% of the esterification reaction is completed within the esterifier. The size (i.e., residence time) and cost of the esterifier for a given plant throughput is determined by the need to accomplish sufficient esterification at the required esterifier reaction conditions (i.e., temperature and feed molar ratio of ethylene glycol to phthalic acid). The esterifier generally can consume more than 70% of the energy input to the system, because the esterifier has to heat the incoming reactants up to the temperature required for the esterification reaction and this reaction requires the vaporization of the water byproduct and excess ethylene glycol used to drive the reaction forward.

Various esterifier designs have been implemented within PET production systems, including thermosiphon esterifiers. Thermosiphon esterifiers generally comprise a vapor separator in combination with a heat exchanger. The heat exchanger can have vertically extending passages for fluid and an upper fluid outlet and a lower fluid inlet, the upper outlet communicating with the side of the thermosiphon system and the lower inlet being connected with the bottom of the thermosiphon system by a conduit loop, overflow means in the vessel for continuous withdrawal of esterification product at a rate which maintains a constant liquid level, means in the upper part of the vessel for withdrawing vapors and means for injecting cold reactant feed mixture into the lower fluid inlet of the heat exchanger. Such esterifier designs are provided, for example, in U.S. Pat. No. 3,927,982 to Chapman and Temple, which is incorporated herein by reference.

### BRIEF SUMMARY OF THE INVENTION

Although thermosiphon esterifiers provide for vigorous mixing and enhanced heat transfer as compared to other types of esterifiers, they can often experience unstable recirculation rates. Such instability can cause operating difficulties in maintaining inventory control within the esterifier. It would thus be advantageous to provide modified esterifier designs to provide increased recirculation rate stability and overall energy savings.

Further, since such systems may operate stably at elevated temperatures, it would be advantageous to utilize the resultant vaporized byproducts produced thereby to provide further efficiency and cost benefit to using such systems.

The present invention provides systems and methods for recycling byproducts (e.g., heat and steam) produced in a thermosiphon esterifier as described herein. Such a method may be applicable to thermosiphon esterifiers used for polyethylene terephthalate) production, which can provide for a more effective PET production process. The inventors have discovered surprising economic benefits associated with the elevated temperatures under which the novel thermosiphon esterifiers described herein may he operated.

The novel thermosiphon esterifier design upon which the steam recycling process is based generally comprises a heat exchange member, a crossover pipe in fluid communication with the heat exchange member, a vapor separation member, and a riser baffle member positioned within the vapor separator and in fluid communication with the crossover pipe. The specific parameters and features of the riser baffle, vapor separator, and remaining components of the thermosiphon esterifier design can vary as described herein in greater detail.

Byproduct steam generated from use of such a thermosiphon esterifier (e.g., as the first stage of a polyethylene terephthalate (PET) production process) can, in certain embodiments, be released from the thermosiphon esterifier at a higher temperature than that released from traditional thermosiphon esterifiers. Byproduct steam generated from use of such a thermosiphon esterifier can, in some embodiments, additionally be released from the thermosiphon esterifier at a higher pressure than that released from traditional thermosiphon esterifiers. Thus, a high value heating product can be withdrawn from the process for other uses.

In one aspect of the invention is provided a method comprising: A. withdrawing steam from a thermosiphon esterifier comprising a heat exchange member, a crossover pipe in fluid communication with the heat exchange member, a vapor separation member, and a riser baffle member positioned within the vapor separator and in fluid communication with the crossover pipe; and B. employing the steam in a process requiring energy input. The withdrawn steam byproduct can be, for example, withdrawn from the vapor separation member. In certain embodiments, employing the steam in such a process can serve to lower the energy required to operate the thermosiphon esterifier (e.g., in the PET production process) as the energy savings from use of the steam can offset the energy that must be added to the system.

In some embodiments, the withdrawn steam is at a temperature of about 105° C. or greater, including about 110° C. or greater, about 120° C. or greater, about 130° C. or greater, about 140° C., from about 105° C. to about 130° C., from about from 105° C. to about 120° C., or from about 120° C. to about 140° C. In some embodiments, the withdrawn steam is at a pressure of about 1.2 bar absolute (barA) or greater, including about 1.5 barA or greater, including about 2.0 barA. or greater, about 3.0 barA or greater, about 4.0 barA, from about 1.2 barA to about 3.0 barA, from about 1.2 barA to about 2.0 bar A, or from about 2.0 barA to about 4.0 barA.

The method described herein can, in some embodiments, further comprise adding reactants through a feed slurry injection port disposed upstream from the heat exchanger. One or more of the reactants may, in certain embodiments, be heated prior to being added, by heat exchange with the steam. The reactants can, for example, comprise ethylene glycol and a phthalic acid, wherein the ethylene glycol and phthalic acid react within the thermosiphon esterifier to produce oligomers.

The energy savings associated with employing the steam can vary and may be, for example, from about 5 to about 100 kJ/kg of oligomer made by the esterifier, from about 10 to about 90 kJ/kg of oligomer made by the esterifier, or from about 20 to about 80 kJ/kg of oligomer made by the esterifier, or can be about 50 kJ/kg of oligomer made by the esterifier or greater. In some embodiments, the process can reduce the amount of wastewater generated from the system, as the water byproduct from esterification (e.g., from PET esterification) can be used in the form of steam for various purposes.

In another aspect of the present disclosure is provided a method for reducing the required energy input for an industrial process, the method comprising: A. operating a thermosiphon esterifier under conditions so as to provide a steam byproduct at a temperature of about 105° C. or greater and a pressure of about 1.5 bar or greater; B. withdrawing the steam byproduct; and C. using the steam byproduct to provide a portion of the required energy input for the industrial process. In some embodiments, the thermosiphon esterifier comprises a heat exchange member, a crossover pipe in fluid communication with the heat exchange member, a vapor separation member, and a riser baffle member positioned within the vapor separator and in fluid communication with the crossover pipe. The steam byproduct can be, for example, withdrawn from the vapor separation member.

In some such embodiments, the steam byproduct is provided at a temperature of about 115° C. or greater and in some such embodiments, the steam byproduct is provided at a pressure of about 2 bar or greater. The industrial process in which the steam byproduct is used can vary; in certain embodiments, the industrial process is a process incorporating the thermosiphon esterifier. Overall, the required energy input for the industrial process can be reduced in varying amounts, such as by about 5% or greater in some embodiments.

### BRIEF DESCRIPTION OF THE DRAWINGS

Having thus described the invention in general terms, reference will now be made to the accompanying drawings, which are not necessarily drawn to scale, and wherein:

FIG. 1 is a schematic illustration of a thermosiphon esterifier having a lightbulb-shaped vapor separator having a riser baffle associated therewith;

FIG. 2 is a schematic illustration of the thermosiphon esterifier of FIG. 1, wherein various parameters of the components are indicated;

FIG. 3 is a schematic cross-section of an exemplary lightbulb-shaped vapor separator having a riser baffle associated therewith;

FIG. 4 is a schematic illustration of a thermosiphon esterifier having a straight sided vapor separator having a riser baffle associated therewith;

FIG. 5 is a schematic illustration of the thermosiphon esterifier of FIG. 4, wherein various parameters of the components are indicated;

FIG. 6 is a schematic cross-section of an exemplary straight sided vapor separator having a riser baffle associated therewith;

FIG. 7 is a schematic illustration of a thermosiphon esterifier having a lightbulb-shaped vapor separator having a riser baffle associated therewith and having a bulge in the thermosiphon pipe;

FIG. 8 is a schematic illustration of the thermosiphon esterifier of FIG. 7, wherein various parameters of the components are indicated;

FIG. 9 is a schematic illustration of a thermosiphon esterifier having a straight sided vapor separator having a riser baffle associated therewith and having a bulge in the thermosiphon pipe; and

FIG. 10 is a schematic illustration of the thermosiphon esterifier of FIG. 9, wherein various parameters of the components are indicated.

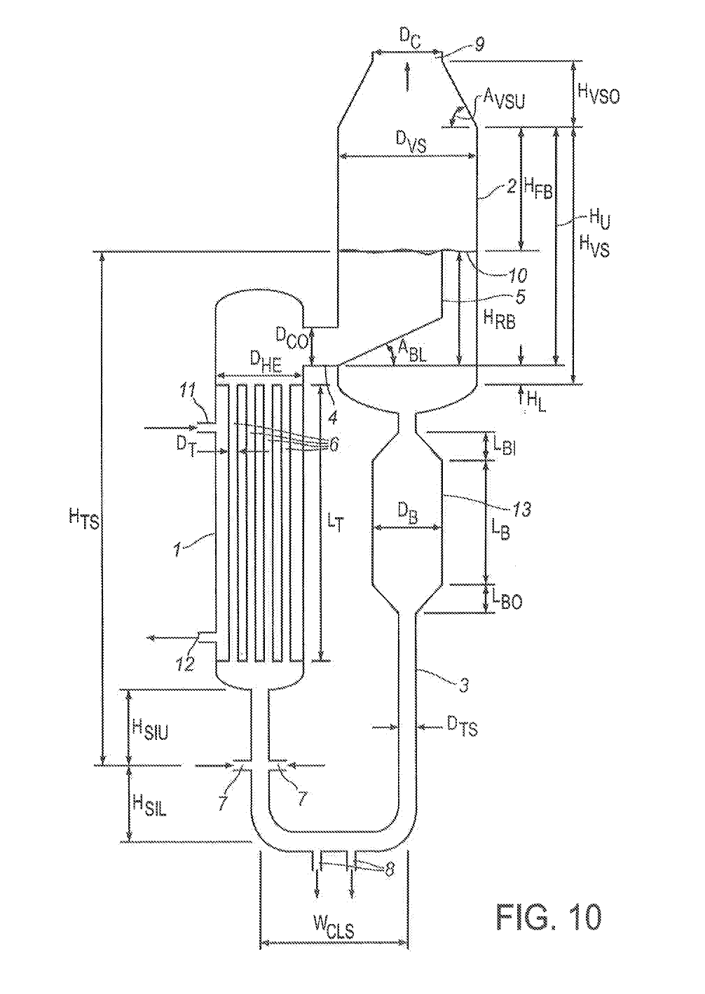

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

Briefly, the present invention provides systems and methods for the production of polyethylene terephthalate (PET). More specifically, the disclosure describes novel esterifier designs that can be used within such systems and methods that can, in some embodiments, provide improved productivity and/or economic benefits. Advantageously, the novel esterifier designs allow fur thermosiphon esterifier operation at higher temperatures and/or higher pressures, which can in some embodiments result in the production (and release) of water-containing byproduct in the form of steam at a relatively high temperature and/or pressure. The details of the novel thermosiphon esterifier are described herein, and various uses of the water-containing byproduct steam produced during use of such a thermosiphon esterifier are provided following such description.

The novel esterifier designs described herein provide improvements over known thermosiphon esterifiers, such as described in U.S. Pat. No. 3,927,982 to Chapman and Temple (hereinafter “the '982 patent), which is incorporated herein by reference, Thermosiphon esterifiers are generally systems for the esterification, e.g., of ethylene glycol and phthalic acids to form oligomers, wherein no mechanical pumping is required to circulate the esterification product.

The thermosiphon esterifiers described in the '982 patent generally comprise an esterification vessel in combination with a heat exchanger, having vertically extending passages for fluid and having an upper fluid outlet and a lower fluid inlet, the upper outlet communicating with the side of the reaction vessel and the lower inlet being connected with the bottom of the reaction vessel by a conduit loop, overflow means in the vessel for continuous withdrawal of esterification product at a rate which maintains a constant liquid level, means in the upper part of the vessel for withdrawing vapors and means for injecting cold reactant feed mixture into the lower fluid inlet of the heat exchanger. In use, the thermosiphon esterifier of the '982 patent is first prefilled with reaction product, heated to reaction temperature, e.g., 250° C. to 400° C., and then a cold slurry of reactants is pumped into the lower fluid inlet of the heat exchanger. In the inlet end of the heat exchanger, the cold reactant slurry feed is quickly mixed with the hot reaction product recycling between the reaction vessel and the heat exchanger, and thereby quickly brought to reaction temperature. Some reactants may vaporize within the heat exchanger and the vaporized components, including water and other volatile byproducts of the esterification reaction evolved in the heat exchanger, can form a liquid comprising a foam. The liquid/foam mixture is less dense than the liquid contained in the thermosiphon pipe on the opposing vertical side of the esterifier. This density difference makes the fluid circulate within the esterifier. Byproduct vapor and excess reactant vapor are removed from the reaction vessel, to maintain a constant pressure, through the means for withdrawing gaseous products, and liquid reaction product is continuously drawn off from the reaction vessel, through the overflow means provided, to maintain a constant liquid level. Typically, two types of recirculating flows are possible, and it is known that in certain situations, a given system can operate between the two types of recirculating flows. The foam in the heat exchanger can expand or collapse, giving the appearance of an overall inventory change in the esterifier. This can also result in instability, making it impossible to change the overall operating pressure within the esterifier significantly.

In certain embodiments, the present disclosure provides a thermosiphon esterifier that can address certain disadvantages associated with traditional thermosiphon esterifiers. Specifically, in some embodiments, a novel thermosiphon esterifier design is provided that can allow for a smaller inventory to be used with the same throughput. In some embodiments, this provides improved productivity as compared with traditional thermosiphon esterifiers (as significantly less residence time in the esterifier may be required, all other reaction conditions being equal). In some embodiments, a higher operating pressure within the thermosiphon esterifier is possible, as thermosiphon recirculation rates may, in some embodiments, not be significantly reduced at higher esterifying pressure. In turn, use of a higher operating pressure can, in certain embodiments, allow the same product to be made with lower excess ethylene glycol in the esterifier, leading to a reduction in plant operating costs. In some embodiments, it is possible to maintain inventory control within the esterifier.

According to one embodiment of the present disclosure, a novel thermosiphon esterifier design is provided, comprising a riser baffle in the vapor separation portion of the thermosiphon esterifier that is adapted to receive product flow from the crossover pipe. The riser baffle can, in some embodiments, reduce the instability commonly associated with the operation of thermosiphon esterifiers. Consequently, in some embodiments, the presence of a riser baffle can help to ensure that the inventory of the esterifier stays consistent. Surprisingly, the incorporation of such a riser baffle can provide a significant improvement to esterifier productivity. In some embodiments, the riser baffle can allow for the use of higher pressures within the thermosiphon esterifier, which can lead to faster reaction times (i.e., less residence time of reactants within the thermosiphon esterifier).

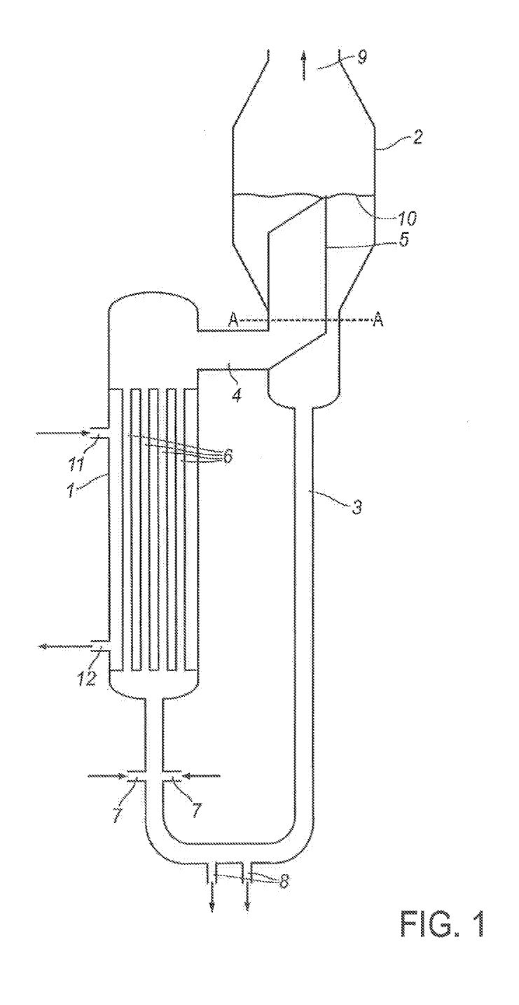

One exemplary thermosiphon esterifier comprising such a riser baffle is illustrated in FIG. 1. The esterifier illustrated in FIG. 1 generally comprises the following elements: heat exchanger 1, vapor separator 2, thermosiphon pipe 3, and crossover pipe 4. A riser baffle 5 is fitted inside the vapor separator 2 and is in fluid communication with the end of the crossover pipe 4. In normal steady state operation, the heat exchanger 1, thermosiphon pipe 3, crossover pipe 4, and riser baffle 5 are filled with pre-made desired esterification product (i.e., PET oligomers) up to the normal liquid level 10. Although this level 10 is depicted as being, roughly halfway up the height of the vapor separator, this is not intended to be limiting and the thermosiphon esterifier can be used with both higher and lower liquid levels. The reactants for the esterification reaction (ethylene glycol and phthalic acids) are fed, as a slurry (at a temperature normally in the range of 70° C. to 150° C., however, the slurry temperature can be higher, such as 200° C. or lower, such as room temperature), into thermosiphon pipe 4 through slurry injection nozzles 7 located below the inlet from thermosiphon pipe 3 to heat exchanger 1. The slurry can comprise the reactants in various molar ratios; for example, the molar ratio of ethylene glycol to phthalic acid can range from about 1:1 to about 4:1. In certain embodiments, the molar ratio of ethylene glycol to phthalic acid is less than or equal to about 2:1 (e.g., between about 1:1 and about 2:1). The injected reactant slurry rapidly mixes with the pre-made oligomer recirculating around the esterifier, effectively heating the reactants to a temperature very near to the temperature required for reaction (i.e., a temperature of at least about 250° C.).

The recirculating oligomer product thus mixes with the freshly injected reactants, which begin to react and the mixture then flows upward through the heat exchanger 1, where the mixture is heated further to reaction temperature by heat transfer fluid contained in heat exchanger tubes 6 (effectively reversing the cooling effect caused by the injection of cool slurry to the recirculating oligomer). The heat transfer fluid is supplied to the heat exchanger 1 through heat transfer fluid inlet 11 and leaves the heat exchanger via the heat transfer fluid outlet 12. During residence within the heat exchanger, much of the excess ethylene glycol reactant added and byproduct water produced in the esterification reaction between ethylene glycol and a phthalic acid are vaporized. The formation of this vapor in heat exchanger 1 reduces the density of the oligomer mixture flowing through the heat exchanger 1. It is this density difference between the vapor-laden oligomer in heat exchanger 1 and the essentially vapor-free oligomer in the down flowing section of the thermosiphon pipe 3, which provides the motive force to drive recirculation of the contents within the thermosiphon esterifier.

After passing through heat exchanger 1, the recirculating reaction mixture (including the oligomer used to pre-fill the system, newly-formed product, excess reactants, and/or byproducts) leaves the heat exchanger via crossover pipe 4, entering vapor separator 2 by flowing upwards through and out over the top of riser baffle 5. As the reaction mixture passes out into vapor separator 2, most of the vapor separates from the oligomer by gravity. The vapor flows upwards and out through vapor outlet 9, while the oligomer flows downwards past riser baffle 5 into the main volume of vapor separator 2.

Vapor separator 2 provides additional residence time for further reaction and allows any vapor formed to flow counter-currently to the downward flowing oligomer. This vapor can exit the system with vapor escaping the oligomer at riser baffle 5. The oligomer flowing through vapor separator 2 exits via thermosiphon pipe 4 connected to the bottom of vapor separator 2. Thermosiphon pipe 4 connects the bottom of vapor separator 2 to the heat exchanger 1 inlet, to allow oligomer to recirculate around the esterifier. Several product discharge nozzles are provided at the lowest point of thermosiphon pipe 4 to allow product to be withdrawn from the esterifier before further slurry injection (e.g., by pumps which transfer oligomer to the for the next step of PET production, polymerization).

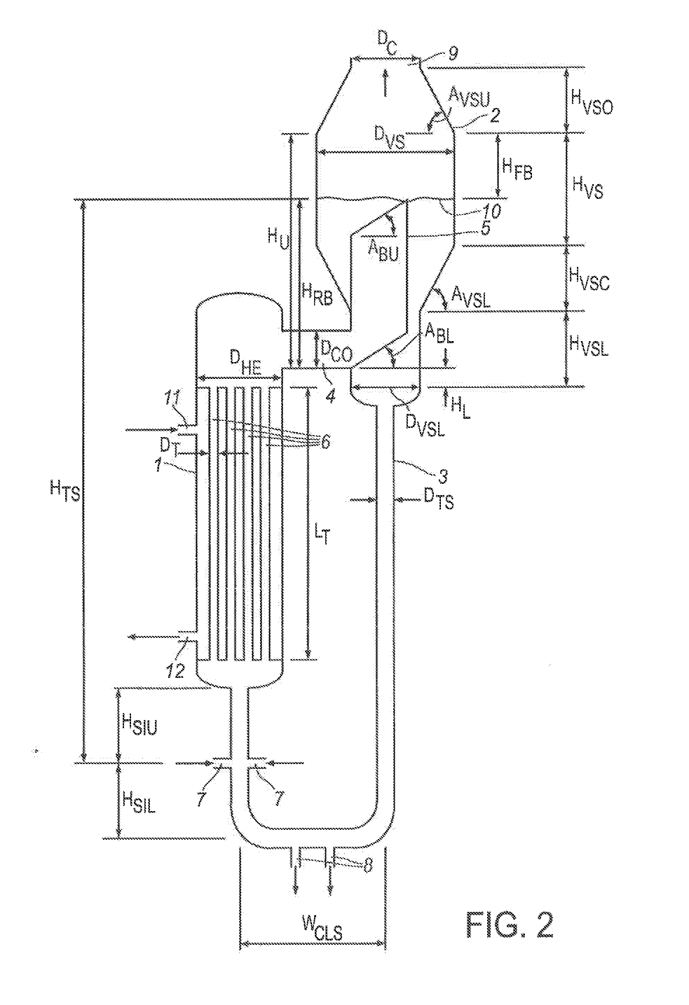

According to the present invention, the inclusion of a riser baffle as described herein can have certain advantages in the operation of a thermosiphon esterifier. A baffle generally is understood to be a fluid flow-directing component. The shape, size, and features of a riser baffle useful according to the present disclosure can vary. For example, in some embodiments, the riser baffle can have a height that is greater than or equal to half the diameter of the crossover pipe (e.g., 4 in the embodiment depicted in FIGS. 1 and 2, having a diameter D_{CO} as shown in FIG. 2) with which it is in contact. In some embodiments, the riser baffle can have a height that is less than the height of the vapor separator unit ((e.g., 2 in the embodiment depicted in FIGS. 1 and 2, having a height H_{U} as shown in FIG. 2). In certain embodiments, the height of the riser baffle can be between these two values. Accordingly, in certain embodiments according to the system configuration in FIGS. 1 and 2, the riser baffle can have a height H_{RB} represented by the formula:

D_{CO}/2≦H_{RB}≦H_{U}.

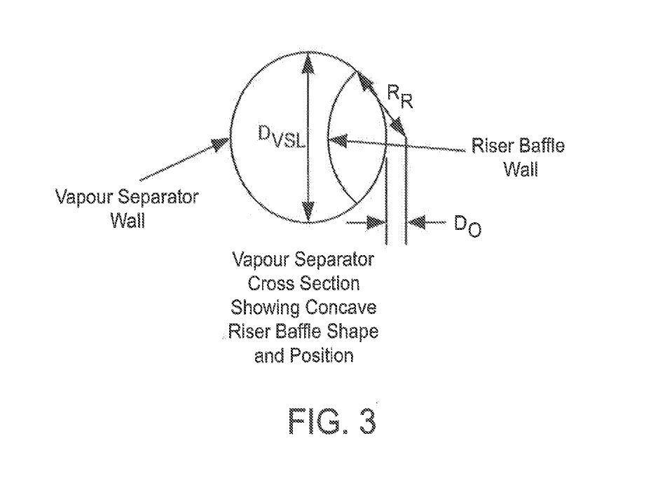

The shape of the riser baffle wall can be convex or concave with respect to the flow from the crossover pipe 4. A cross-sectional view showing the riser baffle 5 of FIGS. 1 and 2 situated within vapor separator 2 is provided in FIG. 3. The outer circle depicts the walls of the vapor separator unit 2. The left most portion of FIG. 3 depicts the cross-sectional area of the riser baffle (upflow region) within the vapor separator unit, with the circular curve in the center of the outer circle depicting the riser baffle wall, which is concave with respect to the inflow from crossover pipe 4. The maximum radius and cross-sectional area of the riser baffle can vary in certain embodiments. The maximum radius and cross-sectional area of the riser baffle, however, must be selected so as to allow for sufficient upflow through the riser baffle and sufficient downflow through the portion of the vapor separator in which the riser baffle is situated to ensure efficient operation of the system as a whole.

In some embodiments, the cross-sectional area of the upflow side of the riser baffle (A_{Ru}) is related to the diameter of the vapor separator above the top of the riser baffle (D^{VS}) by the formula:

0.05π(D_{VS})²/4≦A_{R}u≦0.95π(D_{VS})²/4.

In some embodiments, the cross-sectional area of the upflow side of the riser baffle (A_{Ru}) is related to the diameter of the vapor separator below the bottom of the riser baffle (D_{VSL}) by the formula:

0.05π(D_{VSL})²/4≦A_{Ru}≦0.95π(D_{VSL})²/4.

The bottom of the riser baffle can, in some embodiments, form an angle between 0 and 80 degrees with the horizontal. Similarly, the top of the riser baffle can, in some embodiments, form an angle between 0 and 80 degrees. These angles can be understood with reference to the embodiment of FIG. 2, wherein A_{BL} depicts the angle of the bottom of the riser baffle and A_{BU} depicts the angle at the top of the riser baffle. In the embodiment illustrated in FIGS. 1 and 2, it is noted that the top of the riser baffle is illustrated as angled (i.e., A_{BU}≧0 degrees). This illustration is not intended to be limiting and it is understood that the top of the riser baffle, as well as the bottom of the riser baffle, may be flat (i.e., A_{BU}=about 0 degrees and/or A_{BL}=about 0 degrees) in certain embodiments. In certain embodiments, a riser baffle with a horizontal top may be advantageous as it may cause fewer stresses on the baffle and be less likely to result in fatigue failure than a riser baffle with an angled top.

The specific shape and size of vapor separator 2, within which the baffle described herein is employed, can vary. The overall shape of the vapor separator in the embodiments illustrated in FIGS. 1 and 2 is referred to as a “lightbulb” design. This design can be described as a vessel comprising two cylindrical sections, one vertically disposed above the other and joined to each other by a conical section, where the upper cylindrical section has a larger diameter (e.g., D_{VS}) than the lower cylindrical section (D_{VSL}). The lower cylindrical section is joined to the thermosiphon pipe with a bottom dished or conical end and the upper cylindrical section is joined to the vapor outlet by a top dished or conical end.

The angles of the wails of the lightbulb-shaped vapor separator can be varied to provide a range of specific designs. For example, as shown in FIG. 2, angles A_{VSU} and A_{VSL} can range from about 0° to about 80° with respect to the horizontal. The optionally angled portions of the vapor separator walls can vary in length (with vertical heights indicated on FIG. 2 as H_{VSC} and H_{VSO}). The height of the vertical walls between the angled portions (labeled as “H_{VS}” in FIG. 2) can vary. Further, the overall diameter (e.g., the maximum diameter) of the vapor separator (D_{VS}), the diameter of the base of the vapor separator (D_{VSL}), and the diameter of the vapor separator exit (D_{C}, to vapor outlet 9) can vary.

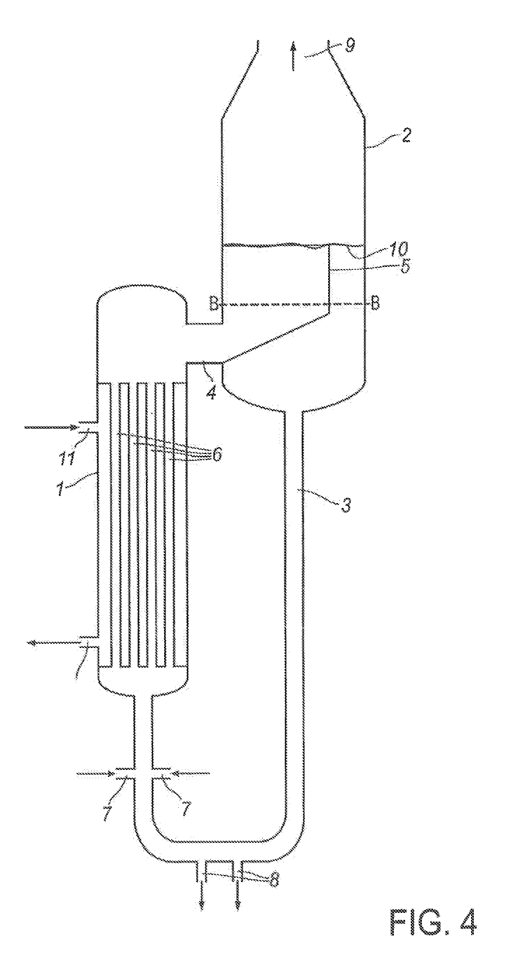

In another embodiment, an alternative vapor separator design within a thermosiphon esterifier, as schematically illustrated in FIG. 4, is provided. As shown in the figure, the geometry of the vapor separator 2 is modified as compared with the vapor separator shown in the esterifier of FIG. 1. Here, the vapor separator has straight sides, which may, in some embodiments, provide additional benefit. For example, such a vessel can provide for a bigger separator without changing the footprint of the overall esterifier significantly. In some embodiments, such a straight-sided vapor separator can provide cost savings benefits in production as it may be simpler to construct. The straight-sided vapor separator in certain embodiments can be described as a cylindrical vessel having a dished or conical end leading to the thermosiphon pipe 3 and a dished or conical end at the top of the vapor separator leading to the vapor outlet.

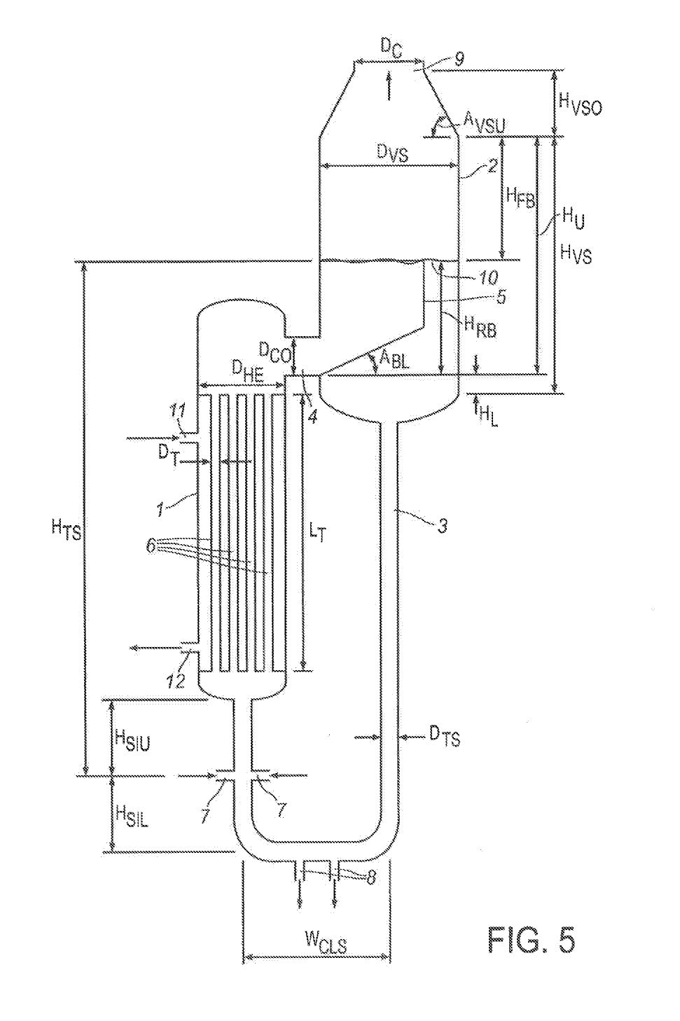

In the embodiment of FIG. 4, the riser baffle 5 is illustrated as having a horizontal top, rather than an angled top as in the embodiment of FIG. 1. Again, this riser baffle geometry is not intended to be limiting; rather, the A_{BL} and A_{BU} angles can vary from 0-80° C. (understood with reference to FIG. 5, wherein depicts various parameters of the system of FIG. 4, including the angle of the bottom of the riser baffle, A_{BU} and the angle at the top of the riser baffle, A_{BL}.

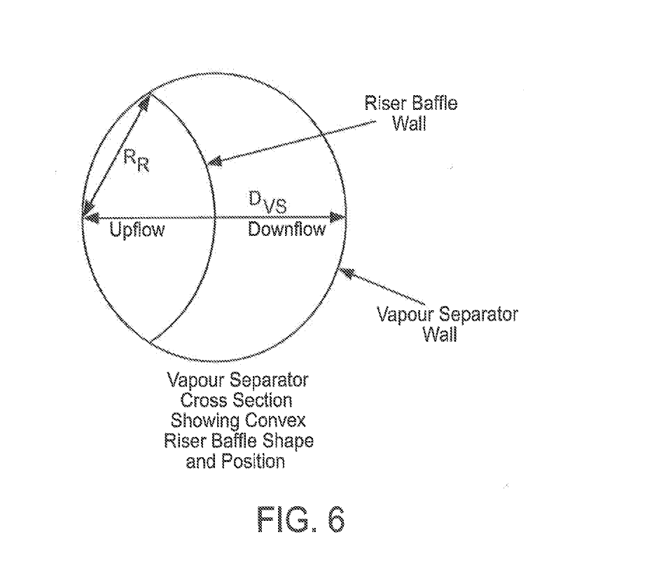

FIG. 6 illustrates a cross-sectional view of an exemplary riser baffle for use in systems employing a straight-sided vapor separator (i.e., as schematically illustrated in FIGS. 4 and 5). In this figure, the outer circle depicts the walls of the vapor separator unit (e.g., 2 in the embodiments of FIGS. 4 and 5). The left most portion of FIG. 6 depicts the cross-sectional area of the riser baffle (upflow region) within the vapor separator unit, with the circular curve in the center of the outer circle depicting the riser baffle wall, which is convex with respect to the in flow from crossover pipe 4. Again, the maximum radius and cross-sectional area of the riser baffle can vary in certain embodiments. The maximum radius and cross-sectional area of the riser baffle, however, must be selected so as to allow for sufficient upflow through the riser baffle and sufficient downflow through the portion of the vapor separator in which the riser baffle is situated to ensure efficient operation of the system as a whole.

The diameter of the vapor separator (either lightbulb or straight-sided design) can be, in some embodiments, selected such that the upward superficial vapor velocity in the vapor separator is less than about 2 meters/second, where the superficial vapor velocity is calculated assuming that the only gases present are steam and ethylene glycol vapor, the esterification reaction is 100% complete, all ethylene glycol in excess of stoichiometric requirements is vaporized and that the gases obey the ideal gas theory. In some embodiments, the vertical distance between the top of the riser baffle and the point at which the vapor separator cross-section starts to reduce (H_{FB}) is between about 0 meters and about 5 meters.

The vapor separator and the baffle feature having been described above, the remaining components of the thermosiphon esterifier and the parameters thereof (e.g., diameters, heights, capacities, distances between components, etc.) can vary. Certain parameters can, however, be beneficial to the operation of the thermosiphon esterifiers described herein. The features described in the present application can be applied to a range of thermosiphon esterifier designs, which may, in some embodiments, provide further benefits as compared with traditional thermosiphon esterifiers.

For example, in some embodiments, there is a minimum vertical distance H_{TS} between the slurry injection point (e.g., 7) and the top of the riser baffle. This height can, in some embodiment, affect the recirculation rate obtained within the thermosiphon esterifier. Advantageously, in some embodiments, a minimal value for this height is about 8 meters or greater (e.g., between about 8 meters and about 20 meters). In some embodiments, there is a preferable range of horizontal distance between the center line of the downflowing leg of the thermosiphon pipe (4) and the center line of the upflowing leg of the thermosiphon pipe, W_{CLS}, which can be defined with respect to the diameter of heat exchanger 1 and the diameter of the vapor separator 2. For example, in some embodiments, this horizontal distance is within a range represented by the following:

(D_{HE}+D_{VS})/2≦W_{CLS}≦(D_{HE}+D_{VS})/2+5),

wherein D_{HE} is the diameter of the heat exchanger (e.g., the maximum diameter of the heat exchanger), and D_{VS} is the diameter (e.g., the maximum diameter) of the vapor separator.

The properties of the heat exchanger 1 and the components thereof can also vary. The heat transfer fluid utilized in the heat exchanger tubes 6 can be one of a number of heat transfer media which can operate up to temperatures of about 340° C. or greater in either the liquid or vapor phase. One exemplary heat transfer fluid used is a mixture of biphenyl and diphenyl oxide, commercially available as DOWTHERM™ A (Dow® Corning Corporation), operating in the vapor phase. In some embodiments, the diameter (D_{T}) of the tubes 6 used in the heat exchanger can be between about 0.5 inches and about 4 inches. The ratio of operating liquid inventory to the heat transfer surface area, based on heat exchanger tube outside diameter, provided by the heat exchanger is, in some embodiments, advantageously greater than about 0.3 cubic meters of operating liquid inventory per square meter of heat transfer surface area.

In some embodiments, a catalyst can be used in the thermosiphon esterifier to promote the reaction. For example, one or more catalysts can be injected into the esterifier with the slurry feed (e.g., into inlet 7). The catalyst can be any type of catalyst known to promote esterification, Oligomerization, and/or polymerization reactions between ethylene glycol and phthalic acids. For example, in certain embodiments, the catalyst can be an organic or inorganic compound (e.g., an antimony, tin, titanium, lanthanum, zinc, copper, magnesium, calcium, manganese, iron, cobalt, zirconium, or aluminum compounds, such as oxides, carbonates, acetates, phosphorus derivatives, alkyls, or alkyl derivatives) or a strong acid (e.g., sulfuric acid, sulfophthalic acid, sulfosalicylic acid, or antimonic acid). See, for example, EP 812818, WO 99/28033; U.S. Pat. No. 6,998,462 to Duan et al., U.S. Pat. No. 3,056,818 to Werber, U.S. Pat. No. 3,326,965 to Schultheis et al.,; U.S. Pat. No. 5,981,690 to Lustig et al.; and U.S. Pat. No. 6,281,325 to Kuruan, which are incorporated herein by reference. In some embodiments, use of a catalyst in the thermosiphon esterifiers described herein can result in increased productivity (e.g., faster reaction, shorter residence time of reactants and products in the esterifier, etc.).

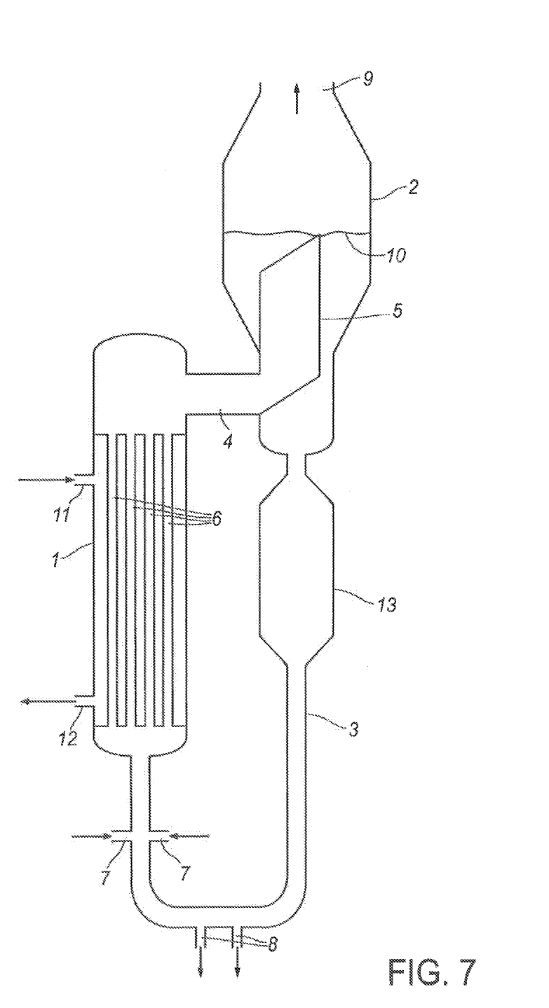

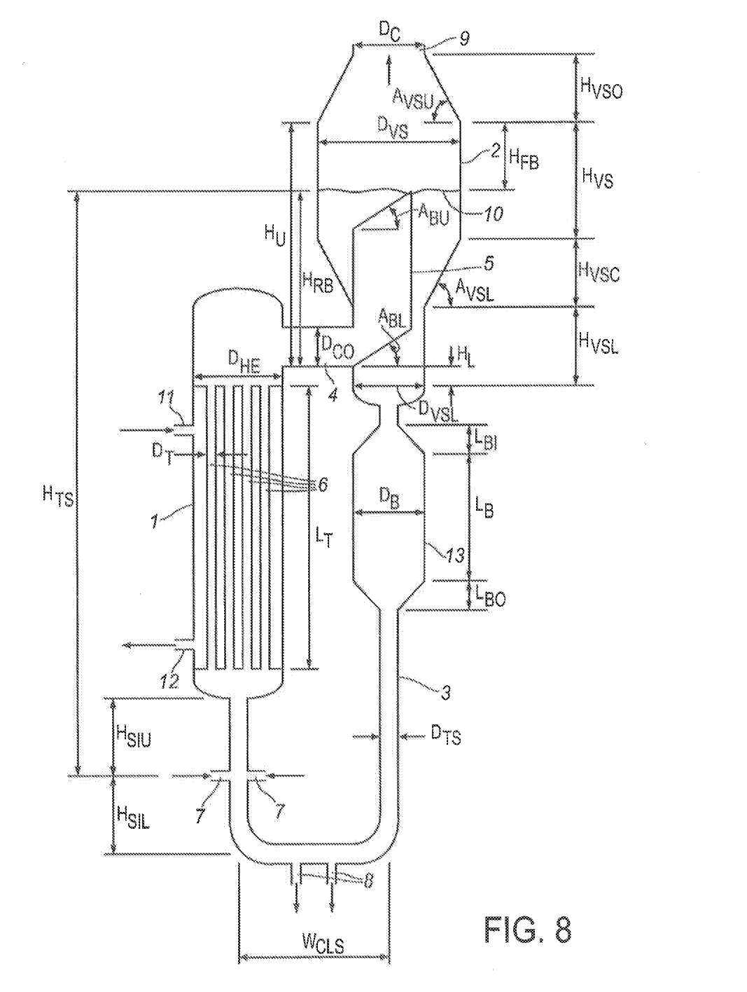

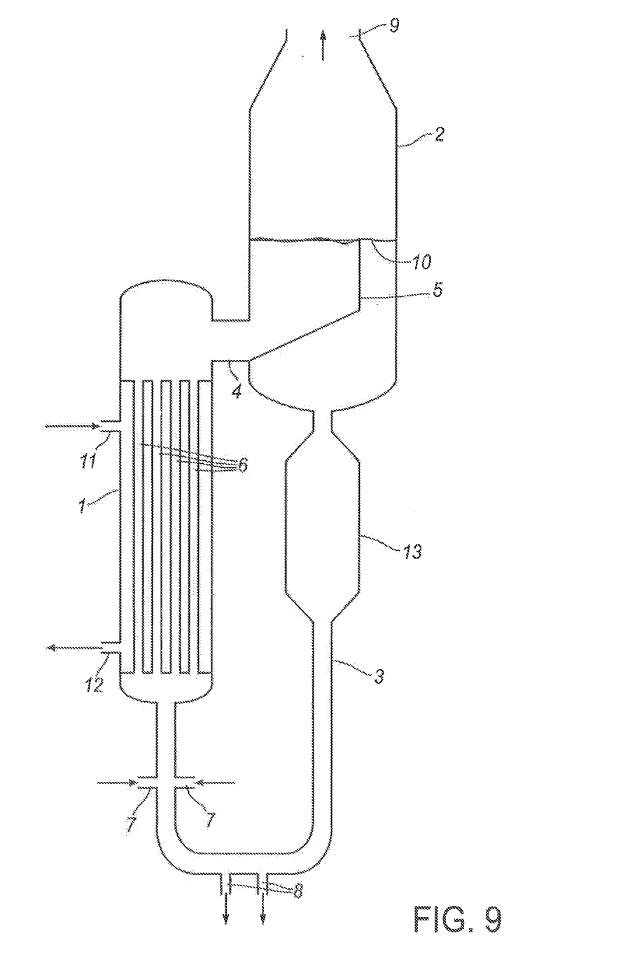

In certain embodiments, the diameter of the crossover pipe 4, having a diameter D_{CO}, has a minimum value, e.g., about 0.2 meters or greater. In some embodiments, the thermosiphon pipe 3 has a minimum diameter, e.g., the diameter of the thermosiphon pipe (D_{TS}) can, in some embodiments, be greater than about 0.2 meters. In certain embodiments, the diameter of the thermosiphon pipe (D_{TS}) can be relatively constant along its length. In certain embodiments, a thermosiphon esterifier according to the present invention comprises a thermosiphon pipe having one or more bulges therein (i.e., portions of the thermosiphon pipe having an enlarged diameter). Exemplary embodiments showing a bulge in the thermosiphon pipe are schematically illustrated in FIGS. 6-7 (wherein the bulge is indicated as Thermosiphon Pipe Bulge 13). FIGS. 6 and 7 illustrate a thermosiphon bulge-containing pipe with a lightbulb-shaped vapor separator, and FIGS. 8 and 9 illustrate a thermosiphon bulge-containing pipe with a straight sided vapor separator. The bulge in the thermosiphon pipe can advantageously serve to increase the operating inventory of the esterifier. Particularly, in some embodiments, the addition of a bulge at this position (i.e., somewhere along the length of the thermosiphon pipe) can provide this added space for esterifier inventory while not significantly impacting the overall footprint of the system. The size and shape of the bulge can vary. In some embodiments, the diameter of the bulge, D_{B}, is greater than or equal to the diameter of the thermosiphon pipe (D_{TS}) but less than or equal to the diameter of the vapor separator (D_{VS}), e.g., the maximum diameter of the vapor separator.

The thermosiphon esterifiers described herein can provide various advantages over traditional thermosiphon esterifiers. In use, the thermosiphon esterifiers of the present disclosure can, in some embodiments, be operated at higher operating pressures than traditional thermosiphon esterifiers and, in some embodiments, thermosiphon esterifier recirculation rates are not significantly reduced at such high operating pressures. This property is in contrast to traditional thermosiphon esterifiers, wherein the greater instability in the esterifier inventory results in greater system instability. In some embodiments, the operating pressure measured in the vapor space at the top of the vapor separator in the thermosiphon esterifiers described herein is greater than about 1.65 atmospheres absolute. Because, in some embodiments, the pressure at which the thermosiphon esterifiers described herein are operated can be increased as compared with traditional thermosiphon operation, a smaller inventory can be used for the same reaction throughput in certain embodiments. In some embodiments, for a given reaction capability, a thermosiphon esterifier as described herein can be smaller in size than a traditional thermosiphon esterifier for the same reaction capability.

The thermosiphon esterifiers described herein can provide various advantages over traditional thermosiphon esterifiers. In use, the thermosiphon esterifiers of the present disclosure can, in some embodiments, be operated at higher operating pressures than traditional thermosiphon esterifiers and, in some embodiments, thermosiphon esterifier recirculation rates are not significantly reduced at such high operating pressures. This property is in contrast to traditional thermosiphon esterifiers, wherein the greater instability in the esterifier inventory results in greater system instability. In some embodiments, the operating pressure measured in the vapor space at the top of the vapor separator in the thermosiphon esterifiers described herein is greater than about 1.65 atmospheres absolute. Because, in some embodiments, the pressure at which the thermosiphon esterifiers described herein are operated can be increased as compared with traditional thermosiphon operation, a smaller inventory can be used for the same reaction throughput in certain embodiments. In some embodiments, for a given reaction capability, a thermosiphon esterifier as described herein can be smaller in size than a traditional thermosiphon esterifier for the same reaction capability.

The thermosiphon esterifiers described herein can be operated batch wise, semi-continuously, or continuously. A continuous process is preferred, wherein the reactants (i.e., terephthalic acid, optionally isophthalic acid for bottle grade polyester resin at a˜less than 5% of the total phthalic acids, and ethylene glycol) can be continuously introduced to the esterifier via inlet 7, and wherein oligomer product can be continuously withdrawn via product discharge outlet 8). Advantageously, the entire thermosiphon esterifier, or at least portions thereof, is insulated to prevent undue heat loss while operating at high temperatures.

In use, the level of liquid on the downflow side of the riser baffle with respect to the cross-over pipe 4 can vary. In the figures, the liquid is illustrated as being above the level of crossover pipe 4 and as being at the same height as the maximum height of riser baffle 5. However, in practice, the liquid level on the downflow side of the riser baffle can be modified significantly and can range from a level below the bottom of the crossover pipe up to a level above the top of the riser baffle (e.g., up to the top of the (upper) cylindrical section of the vapor separator). For example, in some embodiments, the liquid level on the downflow side of the riser baffle can be at about the top of the crossover pipe or below or higher. Although in traditional thermosiphon esterifiers, having the liquid above the top of the crossover pipe can result in excessive vibration of the system and can lead to equipment damage, the novel thermosiphon esterifiers described herein may, in certain embodiments, be operated with varying liquid levels with little to no detrimental effect.

In traditional thermosiphon esterifiers, the water byproduct of the esterification process is generally considered to be a waste product and is thus typically treated and discarded as wastewater. The water byproduct is generally present within the thermosiphon esterifier in the form of steam and is generally removed from the system via a vapor outlet positioned at the top of the vapor separator unit. The water-containing vapor from such systems can optionally be condensed using, e.g., cooling water or ambient air. The steam condensate can then be treated to remove organic materials, producing a stream of almost-pure water, which can then be discharged from the plant as an effluent (i.e., as waste water).

According to the present disclosure, the water byproduct (i.e., steam) generated within the thermosiphon esterifier and withdrawn therefrom via vapor outlet 9 is provided at an elevated temperature and/or elevated pressure. The steam thus can be employed for various purposes to offset the energy usage of the thermosiphon esterifier from which the steam is released. Consequently, the present disclosure provides for the withdrawal of steam at elevated temperature and/or pressure from the thermosiphon esterifier design described herein and further provides for the use of such steam to provide some degree of energy savings to the operation of the thermosiphon esterifier or to provide a further benefit where low cost energy is useful. In particular, the withdrawn steam can provide a source of heating energy and/or energy of expansion (e.g., production of electrical power via a steam turbine) that can be utilized in a variety of end uses.

In some embodiments, the steam byproduct can be used in the context of the continuous polyester polymerization process in which it is produced or in a separate continuous polyester polymerization process. For example, the heated steam can be employed as a heat transfer liquid in a heat exchanger to heat various materials. In one particular embodiment the heated steam can be employed as the heat transfer liquid in a heat exchanger to pre-heat the ethylene glycol and phthalic acid slurry prior to injection of the slurry into the thermosiphon esterifier. The steam from a given thermosiphon esterifier can be used to pre-heat the ethylene glycol and terephthalic acid reactant mixture to be injected into that thermosiphon esterifier or into a different thermosiphon esterifier.

Consequently, in such embodiments, the ethylene glycol and phthalic acid reactant slurry can be introduced into the thermosiphon esterifier at a higher temperature. As such, less energy is required to heat the reactants within the thermosiphon esterifier (e.g., via heat exchanger 2) to ensure that the reactants are at a sufficient temperature for reaction. This provides an energy savings associated with the thermosiphon esterifier system as a whole. The energy savings associated with use of the steam in this way can vary, e.g., from about 5 to about 100 kJ/kg of oligomer made by the esterifier (e.g., from about 10 to about 90 kJ/kg of oligomer made by the esterifier or from about 20 to about 80 kJ/kg of oligomer made by the esterifier). In certain embodiments the energy savings can be about 5 kJ/kg of oligomer made by the esterifier or greater, about 10 kJ/kg of oligomer made by the esterifier or greater, about 5 kJ/kg of oligomer made by the esterifier or greater, about 20 kJ/kg of oligomer made by the esterifier or greater, about 30 kJ/kg of oligomer made by the esterifier or greater, about 40 kJ/kg of oligomer made by the esterifier or greater, about 50 kJ/kg of oligomer made by the esterifier or greater, about 60 kJ/kg of oligomer made by the esterifier or greater, or about 70 kJ/kg of oligomer made by the esterifier or greater.

In another embodiment, the heated steam can be employed as the heat transfer liquid in a heat exchanger to pre-heat one component of the ethylene glycol and phthalic acid reactant slurry prior to injection of the slurry into the thermosiphon esterifier (e.g., to pre-heat just the phthalic acid or to pre-heat just the ethylene glycol). The steam from a given thermosiphon esterifier can be used to pre-heat the terephthalic acid prior to combining it with the ethylene glycol or to pre-heat the ethylene glycol prior to combining it with the terephthalic acid, giving the mixture to be injected into that thermosiphon esterifier or into a different thermosiphon esterifier.

Consequently, in such embodiments, wherein either the ethylene glycol or the phthalic acid is pre-heated prior to forming the reactant slurry to be injected into the thermosiphon esterifier, the resultant reactant slurry can be introduced into the thermosiphon esterifier at a higher temperature. As such, less energy is required to heat the reactants within the thermosiphon esterifier (e.g., via heat exchanger 2) to ensure that the reactants are at a sufficient temperature for reaction. This provides an energy savings associated with the thermosiphon esterifier system as a whole. The energy savings associated with use of the steam in this way can vary, e.g., from about 5 to about 100 kJ/kg of oligomer made by the esterifier (e.g., from about 10 to about 90 kJ/kg of oligomer made by the esterifier or from about 20 to about 80 kJ/kg of oligomer made by the esterifier). In certain embodiments the energy savings can be about 5 kJ/kg of oligomer made by the esterifier or greater, about 10 kJ/kg of oligomer made by the esterifier or greater, about 5 kJ/kg of oligomer made by the esterifier or greater, about 20 kJ/kg of oligomer made by the esterifier or greater, about 30 kJ/kg of oligomer made by the esterifier or greater, about 40 kJ/kg of oligomer made by the esterifier or greater, about 50 kJ/kg of oligomer made by the esterifier or greater, about 60 kJ/kg of oligomer made by the esterifier or greater, or about 70 kJ/kg of oligomer made by the esterifier greater.

In some embodiments, the steam can be used to operate steam vacuum pumps, such as described in U.S. Pat. No. 4,758,650 to Schulz Van Endert, which is incorporated herein by reference. As described therein, steam can be withdrawn and used for producing the vacuum in a vacuum reactor.

Of course, it is to be understood that the steam at an elevated temperature and/or pressure that is produced during use of the novel thermosiphon esterifier design described herein (e.g., during PET production) can advantageously be withdrawn from the thermosiphon esterifier and used for various purposes. The steam can be employed in a variety of manners, such as to produce mechanical motion, or to heat various substances (e.g., through heat exchange). For example, the steam may be directed to turbines, engines, heat exchangers, and the like, and the produced steam can be utilized as a working fluid if desired. In particular, the production of the steam as discussed herein can be useful in that the esterification process as described above can be combined with one or more further industrial processes, and the steam byproduct of the presently described process can be utilized for energy input in the further industrial process. Thus, combinations of industrial processes are encompassed by the present disclosure.

The energy savings arising from such processes and combinations of processes can vary. In certain embodiments, an industrial process employing the withdrawn steam from a thermosiphon esterifier as described herein may benefit from a reduced energy input for operation. For example, the required energy input for the industrial process can be reduced by about 1% or greater, about 2% or greater, about 5% or greater, including from about 1% to about 80%, from about 1% to about from about 1% to about 20%, from about 1% to about 10%, from about 2% to about 80%, from about 2% to about 50%, from about 2% to about 20%, from about 2% to about 10%, from about 5% to about 80%, from about 5% to about 50%, from about 5% to about 20%, and from about 5% to about 10%. Various advantages of use of the steam generated within the riser baffle-containing thermosiphon esterifiers described herein are more evident in the examples provided in the Experimental discussion below.

### EXPERIMENTAL

The experimental data provided herein is based on the embodiments illustrated by the Figures. Computer modeling data based on these embodiments is provided.

### Example 1

A thermosiphon esterifier constructed according to FIG. 1 is operating at a pressure of 2 barA. The steam from the process leaves the top of the esterifier column at a temperature of 120° C. This steam is sent to a heat exchanger which pre-heats the slurry of the ethylene glycol and phthalic acids being fed to the esterifier from 90° C. to 115° C. This reduces the required energy input for the esterifier by ˜72 kJ/kg of oligomer made by the esterifier

### Example 2

A thermosiphon esterifier constructed according to FIG. 1 is operating at a pressure of 2 barA. The steam from the process leaves the top of the esterifier column at a temperature of 120° C. This steam is sent to a heat exchanger which pre-heats the phthalic acid, being fed to an ethylene glycol and phthalic acid slurry mixing vessel, from 20° C. 115° C. The slurry made in the slurry mixing vessel is subsequently fed to the thermosiphon esterifier. This pre-heating of the phthalic acid reduces the required energy input for the esterifier by ˜76 kJ/kg of oligomer made by the esterifier.

### Example 3

A thermosiphon esterifier constructed according to FIG. 1 is operating at a pressure of 3 barA. The steam from the process leaves the top of the esterifier column at a temperature of 133° C. This steam is sent to a heat exchanger which pre-heats ethylene glycol, which is recycled from the polymerization section of a polyester plant and is fed to an ethylene glycol and phthalic acid slurry mixing vessel, from 55° C. to 128° C. The slurry made in the slurry mixing vessel is subsequently fed to the thermosiphon esterifier. This pre-heating of the ethylene glycol reduces the required energy input for the esterifier by ˜62 kJ/kg of oligomer made by the esterifier.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for offsetting energy required for the operation of a thermosiphon esterifier, the method comprising:
A. withdrawing a steam byproduct from a thermosiphon esterifier comprising a heat exchange member, a crossover pipe in fluid communication with the heat exchange member, a vapor separation member, and a riser baffle member positioned within the vapor separator and in fluid communication with the crossover pipe; and
B. using the steam byproduct to provide a portion of the energy required fur the operation of the thermosiphon esterifier.

2. The method of claim 1, wherein the withdrawn steam byproduct is at a temperature of about 105° C. or greater.

3. The method of claim 1, wherein the withdrawn steam byproduct is at a pressure of about 1.2 barA or greater.

4. The method of claim 1, wherein the withdrawn steam byproduct is withdrawn from the vapor separation member.

5. The method of claim 1, further comprising adding reactants through a feed slurry injection port disposed upstream from the heat exchange member.

6. The method of claim 5, further comprising heating one or more of the reactants prior to adding by heat exchange with the withdrawn steam byproduct.

7. The method of claim 1, wherein the reactants comprise ethylene glycol and a phthalic acid, and wherein the ethylene glycol and phthalic acid react within the thermosiphon esterifier to produce oligomers.

8. The method of claim 7, wherein the energy savings associated with using the withdrawn steam byproduct is from about 5 to about 100 kJ/kg of oligomer made by the esterifier.

9. The method of claim 7, wherein the energy savings associated with using the withdrawn steam byproduct is from about 10 to about 90 kJ/kg of oligomer made by the esterifier.

10. The method of claim 7, wherein the energy savings associated with using the withdrawn steam byproduct is from about 20 to about 80 kJ/kg of oligomer made by the esterifier.

11. The method of claim 7, wherein the energy savings associated with using the withdrawn steam byproduct is about 50 kJ/kg of oligomer made by the esterifier or greater.

12. The method of claim 1, wherein the energy required for the operation of the thermosiphon esterifier is reduced by about 5% or greater.

13. A method for reducing the required energy input for an industrial process, the method comprising:
A. operating a thermosiphon esterifier under conditions so as to provide a steam byproduct at a temperature of about 105° C. or greater and a pressure of about 1.2 barA or greater;
B. withdrawing the steam byproduct; and
C. using the steam byproduct to provide a portion of the required energy input for the industrial process.

14. The method of claim 13, wherein the thermosiphon esterifier comprises a heat exchange member, a crossover pipe in fluid communication with the heat exchange member, a vapor separation member, and a riser baffle member positioned within the vapor separator and in fluid communication with the crossover pipe.

15. The method of claim 14, wherein the steam byproduct is withdrawn from the vapor separation member.

16. The method of claim 12, wherein the steam byproduct is provided at a temperature of about 115° C. or greater.

17. The method of claim 12, wherein the steam byproduct is provided at a pressure of about 2 barA or greater.

18. The method of claim 12, wherein the industrial process is a process incorporating the thermosiphon esterifier.

19. The method of claim 12, wherein the required energy input for the industrial process is reduced by about 5% or greater.
